(19) United States
(12) Patent Application Publication
(10) Pub. No.: US 2011/0318822 A1
(43) Pub. Date: Dec. 29, 2011

(54) ANALYTICAL STRIP

(76) Inventors: Chih-Wei Hsieh, Taiwan (CN); Wen-Pin Hsieh, Taiwan (CN); Yi-Jen Wu, Taiwan (CN)

(21) Appl. No.: 12/306,972

(22) PCT Filed: Aug. 29, 2008
(86) PCT No.: PCT/CN08/01551

§ 371 Date: Dec. 30, 2008

(51) Int. Cl. C12M 1/40; G01N 30/96

(52) U.S. Cl. 435/288.7; 422/69

(57) ABSTRACT

This invention discloses an analytical strip comprising a substrate. The substrate has a channel provided concavely on the upper surface thereof. The channel comprises a first area for receiving a fluid sample, a second area for delivering the fluid sample, and a third area where the fluid sample reacts. These three areas are connected sequentially. Nitrocellulose layers are formed at the bottoms of both the second area and the third area. Each of the nitrocellulose layers comprises a hollow-matrix conformation. In addition, the nitrocellulose layer of the second area has an average thickness that is not greater than that of the nitrocellulose layer of the third area. The strip also comprises a reaction material formed in the hollow-matrix conformation of each of the nitrocellulose layers.

### BACKGROUND OF THE INVENTION

1. Technical Field

The present invention relates to analytical strips, and more particularly to an analytical strip for biochemical or immunological assays.

2. Description of Related Art

Conventional analytical strips used in biochemical or immunological assays usually have a substrate or a baseboard provided with a channel or a microfluidic channel. While such channel is typically bordered by a non-absorptive material, and the viscosity of the fluid sample to be analyzed is usually high for the sample is mainly composed of proteins or carbohydrates, part of the fluid sample tends to adhere to the surface of the channel and will not be reacted. Such scenario, if happens, will not only disadvantageously cause the waste of the fluid sample to be analyzed, but also will adversely affects the accuracy of quantifying assays.

In addition, the conventional analytical strip may facilitate the flow of the fluid sample by microfluidic channels so that the fluid sample will be delivered via the capillary force exerted by the structures of such channels to the reaction area. Another alternative approach to deliver the fluid sample involves applying a driving force, such as by a pressurizing means, at the time the fluid sample is introduced into the channel so that the fluid sample is propelled to the reaction area through the channel. However, either one of the aforementioned approaches tends to cause air bubbles occurring after the fluid sample is introduced into the channel. These bubbles, either large or small, will block the channel and result in inaccurate analyzing results.

Furthermore, the manufacturing process of the channels or microfluidic channels on the current substrates is usually involves molding, injection forming or imprinting. Consequently, the analytical strips comprising those above-mentioned substrates have to be made of high-priced micro-injection molds manufactured by using micro-machining or LIGA (abbreviation of “Lithographie GalVanoformung Abformung”, or “Lithography Electroforming Micro Molding” in english) technique. The micro-injection mold used in the manufacturing process tends to wear out rapidly, which results in the relatively high cost.

### SUMMARY OF THE INVENTION

In an attempt to overcome the recited drawbacks and shortcomings of the conventional analytical strips, the present invention provides an analytical strip that comprises a substrate having a channel provided concavely on an upper surface of the substrate. The channel comprises a first area for receiving a fluid sample, a second area for delivering the fluid sample, and a third area where the fluid sample reacts. These three areas are connected sequentially. The analytical strip is characterized in that nitrocellulose layers are formed at each bottom of both the second and the third area, and the conformation of the nitrocellulose layers is a hollow matrix. In addition, the nitrocellulose layer of the second area has an average thickness that is not greater than the thickness of the nitrocellulose layer of the third area. The analytical strip also comprises a reaction material formed in the hollow matrices of the nitrocellulose layers.

Hence, a primary object of the present invention is to provide an analytical strip that has a thin absorptive nitrocellulose layer on the bottom of channel. The thin absorptive nitrocellulose layers act as sample delivering and/or separating function. The channel thus has lower residual of samples in contrast to the traditional microfluidic channel, and low volume of samples needed for multi-analytes detection in a test is realized.

Another object of the present invention is to provide an analytical strip that comprises absorptive nitrocellulose layers having a constant volumetric absorptive capacity and thus allows a quantitative assay to be conducted via controlling the volume of the nitrocellulose layers.

Still another object of the present invention is to provide an analytical strip that has absorptive nitrocellulose layers with a hollow-matrix configuration, which is capable of destroying the air bubbles in the fluid sample when the fluid sample flows through the hollow matrix, as well as preventing the bubbles from blocking the channel or the microfluidic channel of the substrate. Thus, an accurate result of the quantitative assay could be assured.

### BRIEF DESCRIPTION OF THE DRAWINGS

The invention as well as a preferred mode of use, further objects and advantages thereof will be best understood by reference to the following detailed description of illustrative embodiments when read in conjunction with the accompanying drawings, wherein:

FIG. 1A is a perspective view of an analytical strip according to a first embodiment of the present invention;

FIG. 1B is a cross-sectional view of the analytical strip according to the first embodiment of the present invention;

FIG. 2A is a perspective view of an analytical strip according to a second embodiment of the present invention; and

FIG. 2B is a cross-sectional view of the analytical strip according to the second embodiment of the present invention.

### DETAILED DESCRIPTION OF THE PREFERRED EMBODIMENTS

While the present invention proposes an analytical strip, the physical and chemical principles, as well as solution applying techniques it employs are known to one skilled in the art and need not be discussed at any length herein. Meanwhile, the accompanying drawings referred to in the following description are provided for illustrative purposes and need not to be made to scale.

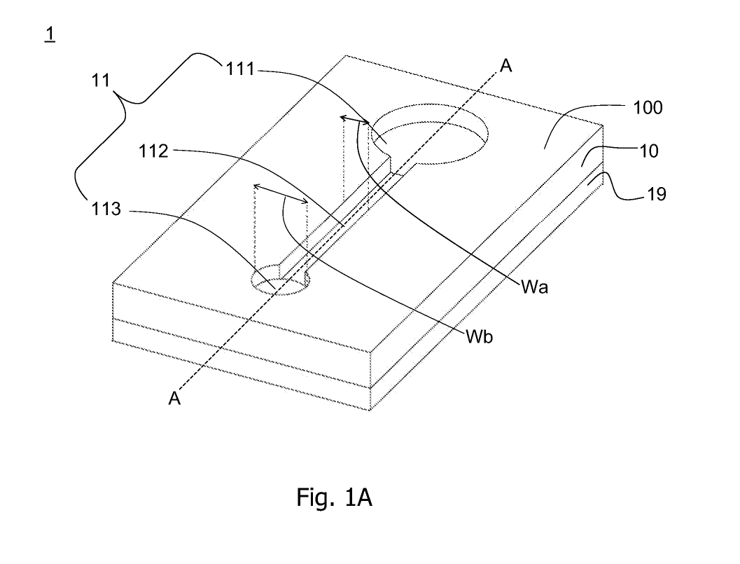

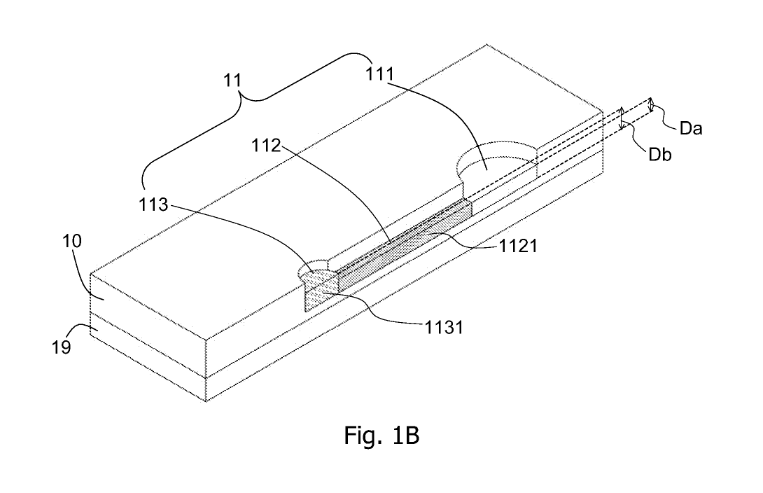

FIG. 1A is a perspective view of an analytical strip according to a first embodiment of the present invention. The analytical strip 1 comprises a substrate 10 and a backing plate 19. The substrate 10 has an upper surface 100 concavely provided with a channel 11. The channel 11 includes a first area 111, a second area 112 and a third area 113 that are connected sequentially. The first area 111 is to receive a fluid sample to be analyzed. The fluid sample is introduced to the first area 111 and then delivered by the second area 112 to the third area 113 where analytes of the fluid sample react and are then detected. In a preferred mode of the present invention, the substrate 10 is made of a biocompatible material.

Now please refer to FIG. 1B, which is a cross-sectional view of the analytical strip 1 taken along Line A-A of FIG. 1A. As shown in FIG. 1B, the nitrocellulose layers 1121 and 1131 are formed respectively at both bottoms of the second area 112 and the third area 113. Each of the nitrocellulose layers 1121 and 1131 has a hollow-matrix conformation that contains a reaction material therein. The composition of the reaction material varies from the category of the analyte in the fluid sample to be detected. The porous hollow matrix serves to absorb the fluid sample coming from the first area 111 and the analytes in the fluid sample can react with the pre-embedded reagents in the nitrocellulose layer 1131. Since the nitrocellulose layers 1121 and 1131 are absorptive, the channel 11 thus has lower residual of samples in contrast to the traditional microfluidic channel, and low volume of samples needed for multi-analytes detection in a test is realized. In addition, when the fluid sample flows through the nitrocellulose layers 1121 and 1131, the hollow-matrix conformation will destroy the air bubbles in the fluid sample, thereby preventing the bubbles from blocking the channel 11.

The analytical strip 1 of the present invention can be applied to either biochemical assays or immunological assay. To detect different analytes of the physiological fluid needs different assays, and different categories of assays require different kinds of reaction materials, which result in different categories of signals. A biochemical quantitative assay, for example, is usually carried out via the enzymatic reaction of the analytes in the biological fluid sample and a chemical luminating reagent, which is catalyzed by the suitable enzymes, to generate optical signals with specific wavelengths for detection. Accordingly, the reaction materials of the analytical strip 1, when applied to the biochemical quantitative assay, will mainly comprise enzymes and the corresponding chemical reagents. On the other hand, when the scenario comes to the quantitative detection of a certain protein in the physiological fluid sample, such as a-fetoprotein, the analytical assay usually utilize a antibody which can specifically recognize the targeted protein and other corresponding chemical reagents to generate detectable signals. Accordingly, the reaction materials of the analytical strip 1, when applied to the quantitative immunoassay, will mainly comprise antibodies and the corresponding reagents. Therefore, the analytical strip 1 of the present invention is adaptive to quantitative detection of various analytes in different types of physiological fluidic specimens (e.g., urine and blood).

Please refer to FIG. 1B, the nitrocellulose layer 1121 of the second area 112 has an average thickness Da which is not greater than the average thickness Db of the nitrocellulose layer 1131 of the third area 113, namely that Da is smaller than or equals to Db. Furthermore, in order to reduce the volume of the fluid sample required, the width Wa of the second area 112 and the width Wb of the third area 113 (shown in FIG. 1A) are both preferably 0.3 mm at least.

The nitrocellulose layers 1121 and 1131 are formed as the following steps. Firstly, a nitrocellulose powder is mixed with an organic solvent containing esters and ketones to form a nitrocellulose solution. The nitrocellulose solution is then applied to the bottoms of the second and third areas 112 and 113 via a casting process. After drying, the nitrocellulose layers 1121 and 1131 are formed respectively at the bottoms of the second areas 112 and third area 113. For a better result of the casting process, the surface roughness (Ra) of the channel 11 preferably ranges from about 3 μm to about 50 μm.

As previously described, after drying the nitrocellulose solution applied onto the bottoms of the second areas 112 and third area 113 forms the nitrocellulose layers 1121 and 1131 which both have the hollow-matrix conformation. In order to obtain a hollow matrix with a better structure, the nitrocellulose powder preferably has a volume that is about nine times to the volume of the organic solvent containing esters and ketones. Because each volumetric unit of nitrocellulose has a constant absorptive capacity, the required volume of the nitrocellulose solution can be derived from the desired volume of the fluid sample to be adsorbed and analyzed before casting. As a result, the required volume of the fluid sample of the analytical strip 1 will be fixedly set, so that the resultant analytical strip 1 is suitable for an assay in a small volume.

After the nitrocellulose layers 1121 and 1131 are solidified at the bottom of the second and third areas 112 and 113, a reaction solution containing reaction materials is injected into the nitrocellulose layers 1121 and 1131, followed by another drying process, such as air-drying or lyophilization. The reaction materials dried in the nitrocellulose layers 1121 and 1131 will be in the form of powder.

As described previously, the reaction materials are added after the solidification of the nitrocellulose layers 1121 and 1131 and then dried therein. Alternatively, the nitrocellulose layers 1121 and 1131 and the reaction materials therein can be formed simultaneously. The nitrocellulose solution that contains the nitrocellulose powder and the organic nitrocellulose solution composed of esters and ketones can mix with the reaction solution having the reaction material in advance, before casting onto the bottoms of the second and third areas 112 and 113. After the drying process (ie, air-drying or lyophilization), the nitrocellulose layers 1121 and 1131 are solidified while the reaction materials are left therein in the form of powder.

The above-described first embodiment of the present invention is an analytical strip having a substrate comprising a channel having three areas. Based on the concept of the present invention, the channel may further comprise a fourth area for accommodating excessive fluid sample introduced into the channel. A second embodiment of the present invention given below is an analytical strip that has a channel including four areas.

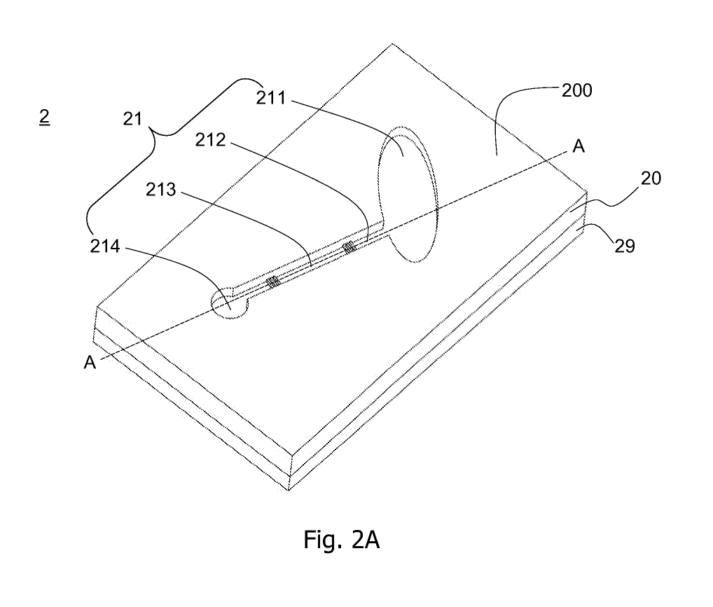

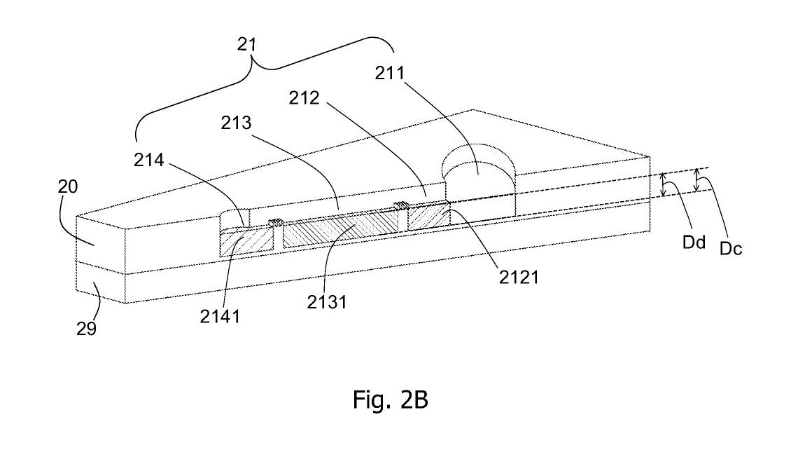

FIG. 2A is a perspective view of the analytical strip according to the second embodiment of the present invention. A substrate 20 of the analytical strip 2 has an upper surface 200 concavely provided with a channel 21. The channel 21 includes a first area 211 for receiving a fluid sample to be analyzed, a second area 212 for delivering the fluid sample, a third area 213 and a fourth area 214. These four areas 211, 212, 213 and 214 are connected sequentially. The fluid sample is introduced to the first area 211 and then delivered via the second area 212 to the third area 213 where the analytes of the fluid sample are reacted.

Please refer to FIG. 2B, which is a cross-sectional view of the analytical strip 2 taken along Line A-A of FIG. 2A. Nitrocellulose layers 2121 and 2131 are formed at bottoms of the second area 212 and the third area 213 respectively. In addition, the nitrocellulose layer 2121 of the second area 212 has an average thickness Dc that equals to the average thickness Dd of the nitrocellulose layer 2131 of the third area 213. Similar to the second and third areas 212 and 213, a nitrocellulose layer 2141 is also formed at the bottom of the fourth area 214 and also has a hollow-matrix conformation for accommodating the excess fluid sample. The nitrocellulose layer 2141 at the bottom of the fourth area 214 is made in the same way as the nitrocellulose layers 2121 and 2131. Namely, the nitrocellulose layers 2121, 2131 and 2141 are formed by casting a nitrocellulose solution onto the bottoms of the second, third and fourth areas 212, 213, and 214, followed by a drying process.

Moreover, The reaction materials can either be formed after the solidification of the nitrocellulose layers 2121, 2131 and 2141, or, alternatively, be formed simultaneously with the nitrocellulose layers 2121, 2131 and 2141 via the similar processes described in the first embodiment. The reaction materials in the nitrocellulose layers 2121, 2131 and 2141 is also in the form of powder.

In addition, in the second embodiment, the structure, dimensions and connective relationships of the first, second and third areas, the preferred material of the substrate, the surface roughness of the channel, the conformation and forming method of the nitrocellulose layers, the preferred ingredients of the nitrocellulose solution and the preferred ratio therebetween, and the preferred composition of the reaction material are all similar to those described in the first embodiment of the present invention and need not to be described herein in further detail.

The present invention has been described with reference to the preferred embodiments and it is understood that the embodiments are not intended to limit the scope of the present invention. Moreover, as the contents disclosed herein should be readily understood and can be implemented by a person skilled in the art, all equivalent changes or modifications which do not depart from the concept of the present invention should be encompassed by the appended claims.

What is claimed is:

1. An analytical strip, primarily comprising a substrate having a channel provided concavely on an upper surface thereof, wherein said channel comprises a first area for receiving a fluid sample, a second area and a third area and these three areas are connected sequentially, the analytical strip being characterized in that:
at the bottom thereof, each of the second and the third area comprises a nitrocellulose layer having a hollow-matrix conformation, wherein the second area is for delivering the fluid sample and the third area is where the fluid sample reacts, and wherein the nitrocellulose layer of the second area comprises an average thickness which is not greater than that of the nitrocellulose layer of the third area; and
a reaction material is formed in the hollow-matrix conformation of each of the nitrocellulose layers.

2. The analytical strip of claim 1, wherein the average thickness of the nitrocellulose layer of the second area is smaller than that of the nitrocellulose layer of the third area.

3. The analytical strip of claim 2, wherein both the nitrocellulose layers of the second and third areas are formed by casting a nitrocellulose solution onto both bottoms of the second and third areas, and then being followed by a drying process.

4. The analytical strip of claim 3, wherein the nitrocellulose solution is a mixture of nitrocellulose powder and an organic solvent containing esters and ketones.

5. The analytical strip of claim 4, wherein the nitrocellulose powder is mixed with the solvent containing esters and ketones at a volumetric ratio of 1:9.

6. The analytical strip of claim 2, wherein each of the second area and the third area has a width of at least 0.3 mm.

7. The analytical strip of claim 2, wherein the substrate is made of a biocompatible material.

8. The analytical strip of claim 2, wherein the channel has a surface roughness ranging from 3 μm to 50 μm.

9. The analytical strip of claim 3, wherein the reaction material in the hollow-matrix conformation is in a powder form and formed by adding a reaction solution containing the reaction material into the nitrocellulose layers, followed by a drying process.

10. The analytical strip of claim 3, wherein the reaction material in the hollow-matrix conformation is in a powder form and formed by mixing a reaction solution containing the reaction material with the nitrocellulose solution and then casting onto the bottoms of the second and third areas, followed by a drying process, so that the nitrocellulose solution forms the nitrocellulose layers while the reaction material dried in the powder form and left in the nitrocellulose layers.

11. The analytical strip of claim 2, wherein the reaction material comprises an enzyme and a chemical reagent.

12. The analytical strip of claim 2, wherein the reaction material comprises an antibody and a chemical reagent.

13. The analytical strip of claim 2, wherein the average thickness of the nitrocellulose layer of the second area equals to that of the nitrocellulose layer of the third area.

14. The substrate of claim 13, wherein the channel further comprises a fourth area having a nitrocellulose layer which is formed at the bottom thereof and also has a hollow-matrix conformation for accommodating excess of the fluid sample.

15. The analytical strip of claim 14, wherein the nitrocellulose layers are formed by casting a nitrocellulose solution onto the bottoms of the second area, the third area and the fourth area followed by a drying process.

16. The analytical strip of claim 14, wherein each of the second area and the third area has a width of at least 0.3 mm.

17. The analytical strip of claim 14, wherein the substrate is made of a biocompatible material.

18. The analytical strip of claim 14, wherein the channel has a surface roughness ranging from 3 μm to 50 μm.

19. The analytical strip of claim 15, wherein the reaction material in the hollow-matrix conformation is in a powder form and is formed by adding a reaction solution containing the reaction material into the nitrocellulose layers, followed by a drying process.

20. The analytical strip of claim 15, wherein the reaction material in the hollow matrices is in a powder form and is formed by mixing a reaction solution containing the reaction material with the nitrocellulose solution and then casting onto the bottoms of the second area, the third area and the fourth area, followed by a drying process, so that the nitrocellulose solution forms the nitrocellulose layers while the reaction material becomes powder contained in the nitrocellulose layers.
